(19) United States
(12) Patent Application Publication
(10) Pub. No.: US 2022/0370190 A1
(43) Pub. Date: Nov. 24, 2022

(54) DEVICES AND METHODS FOR DELIVERING BLOOD FROM A LOWER PRESSURE REGION TO A HIGHER PRESSURE REGION

(71) Applicant: CARDIOST, INC., Denver, CO (US)

(72) Inventors: Fernando ANZELLINI, Bogota (CO); Nicolás ANZELLINI, Denver, CO (US)

(21) Appl. No.: 17/746,180

(22) Filed: May 17, 2022

Related U.S. Application Data
(60) Provisional application No. 63/189,896, filed on May 18, 2021.

(51) Int. Cl. A61F 2/06; A61B 17/00; A61B 17/11

(52) U.S. Cl. CPC A61F 2/06; A61B 17/00234; A61B 17/11; A61B 2017/1107

(57) ABSTRACT

A device and method for diverting a portion of oxygenated blood from a lower pressure region, e.g., left atrium or pulmonary vein, and providing it to the aorta, bypassing the left ventricle, operating at least in part, on the Venturi effect. The device includes a first conduit that diverts a portion of blood from the aorta to a parallel flow path. The device includes a second conduit that delivers blood from the lower pressure region to the first conduit. The blood from the lower pressure region in the second conduit is combined with the blood from the aorta in the first conduit and returned to the aorta. The second conduit is coupled to the first conduit at or near a narrow segment of the first conduit. A Venturi effect at or near the narrow segment draws the blood from the lower pressure region into the first and/or second conduit.

### CROSS REFERENCE TO RELATED APPLICATIONS

This application claims the benefit of priority pursuant to 35 US.C. § 119(e) of U.S. Provisional Patent Application No. 63/189,896, filed May 18, 2021, titled “Devices and Methods for Delivering Blood From a Lower Pressure Region to a Higher Pressure Region,” which is hereby incorporated by reference in its entirety.

### TECHNICAL FIELD

The present disclosure pertains to devices and methods for delivering blood from a lower pressure region to a higher pressure region. In particular, the present disclosure pertains to devices and methods for delivering oxygenated blood from a chamber of a heart to a blood vessel.

### BACKGROUND

The left ventricle of the heart is responsible for pumping oxygenated blood received from the lungs to the aorta to deliver the oxygenated blood to the rest of the body. The left ventricle receives oxygenated blood from the lungs via the left atrium (auricle). The left ventricle of a healthy heart has an ejection fraction of approximately 50 to 70 percent. In other words, with each heartbeat, the left ventricle provides 50 to 70 percent of blood received from the left atrium to the aorta. When the ejection fraction of the left ventricle falls below 50 percent, the heart is no longer efficiently providing oxygenated blood to the rest of the body. Low ejection fraction may be associated with one or more diseases or disorders such as congenital heart defects, cardiomyopathy, diabetes, coronary artery disease, myocardial infarction, and uncontrolled high blood pressure. Subjects with low ejection fractions may suffer health effects such as fatigue, weakness, edema, shortness of breath, and mental confusion. Depending on the severity, low ejection fraction may lead to reduced quality of life or death if left untreated.

In some cases, the underlying cause of the low ejection fraction of the left ventricle can be corrected, for example, by repairing or replacing a malfunctioning mitral valve. However, in other cases, such as when the cardiac muscle of the left ventricle is weakened or damaged, the cause of the low ejection fraction cannot be corrected or reversed. In these instances, particularly for severe cases, a subject may require a heart transplant, an artificial heart, or a pump that assists the left ventricle. The availability of donated hearts for transplants is very low, recipients are required to remain on immunosuppressant drugs with varying levels of side effects, and even with these drugs, there is still the potential for organ rejection by the recipient. Artificial hearts and pumps, while more readily available, require multiple moving parts (e.g., pumps, motors), which require power sources such as batteries. Moving parts may be more prone to failure than static parts, and power sources require external charging and/or periodic replacement. Thus, the subject may be at risk of adverse health outcomes due to failure of a moving part or power source. The subject may also require secondary surgeries to replace parts and/or power sources. Accordingly, improved techniques for compensating for low ejection fraction of the left ventricle are desired.

### SUMMARY

Devices and methods are disclosed for delivering blood from a lower pressure region to a higher pressure region. In some applications, the devices and methods may not require a power source and/or moving parts. In some applications, the devices and methods may divert a portion of oxygenated blood from a left atrium or a pulmonary vein and deliver the blood to the aorta, without passing through the left ventricle. Delivering oxygenated blood from the left atrium or pulmonary vein to the aorta may at least partially compensate for low ejection fraction of the left ventricle.

According to at least one example disclosed herein, a device may include a first conduit with a first end having a first diameter, a second end opposite a first end and having a second diameter, the first end and the second end being configured to be fluidly coupled to the higher pressure region, and a narrow segment disposed between the first end and the second end and having a third diameter, such that the third diameter is less than the first diameter and the second diameter; and a second conduit comprising a third end configured to be fluidly coupled to the lower pressure region, and a fourth end opposite the third end, the fourth end being coupled to the first conduit at or near the narrow segment of the first conduit.

According to at least one example disclosed herein, a method may include diverting, through a first conduit, a first portion of blood from the higher pressure region, the first end of the first conduit being fluidly coupled to the higher pressure region at a first location and a second end of the first conduit being fluidly coupled to the higher pressure region at a second location downstream of the first location; diverting, through a second conduit, a second portion of blood from the lower pressure region, the third end of the second conduit being fluidly coupled to the lower pressure region and a fourth end of the second conduit being fluidly coupled to a narrow segment of the first conduit, the narrow segment disposed between the first end and the second end; drawing the second portion of blood from the second conduit into the first conduit; and providing the first portion of blood and the second portion of blood to the higher pressure region at the second end of the first conduit.

### BRIEF DESCRIPTION OF THE DRAWINGS

FIG. 1 is an illustration of a device coupled to a heart and an aorta according to at least one embodiment of the present disclosure.

FIG. 2 is an illustration of a portion of the device illustrated in FIG. 1.

FIG. 3 is an illustration of a portion of the device illustrated in FIGS. 1 and 2.

FIG. 4 is an illustration of a device coupled to a heart and an aorta according to at least one embodiment of the present disclosure.

FIG. 5 is a flow chart of a method according to at least one embodiment of the present disclosure.

FIG. 6 is a flow chart of a method according to at least one embodiment of the present disclosure.

### DETAILED DESCRIPTION

The following description of certain embodiments is merely exemplary in nature and is in no way intended to limit the disclosure or its applications or uses. In the following detailed description of embodiments of the present devices, apparatuses, systems, and methods, reference is made to the accompanying drawings which form a part hereof, and which are shown by way of illustration specific embodiments in which the described devices, apparatuses, systems, and methods may be practiced. These embodiments are described in sufficient detail to enable those skilled in the art to practice presently disclosed devices, apparatuses, systems, and methods and it is to be understood that other embodiments may be utilized and that structural and logical changes may be made without departing from the spirit and scope of the present disclosure. Moreover, for the purpose of clarity, detailed descriptions of certain features, such as well-known anatomical structures and medical conditions, will not be discussed when they would be apparent to those with skill in the art so as not to obscure the descriptions of the present disclosure. The following detailed description is therefore not to be taken in a limiting sense, and the scope of the present devices, apparatuses, systems, and methods is defined only by the appended claims.

A healthy left ventricle must generate a significant level of pressure when it contracts (e.g., systole), and thereby ejects oxygenated blood, in order to generate sufficient flow of blood through the body. Pressure in the aorta, which receives the oxygenated blood from the left ventricle, is also high, particularly during ventricular systole. In contrast, the left atrium, which receives oxygenated blood from the lungs via the pulmonary veins and provides the blood to the left ventricle, has a lower pressure than the left ventricle and aorta during ventricular systole.

Current techniques for compensating for low ejection fraction of a compromised left ventricle include a pump that may assist the left ventricle (e.g., squeeze the left ventricle or inflate a balloon within the left ventricle during ventricular systole to increase pressure generated by the left ventricle) to deliver oxygenated blood to the aorta. However, current techniques require the pump to be powered (e.g., by electricity) in order to provide adequate pressure to deliver blood from the left ventricle to the aorta. The pump increases the pressure in the left ventricle to be greater than the pressure in the aorta in order to deliver the oxygenated blood from the left ventricle to the aorta during ventricular systole. There are currently no techniques for delivering blood from a lower pressure region (e.g., the left atrium, pulmonary vein) to a higher pressure region (e.g., aorta) without a power source and moving parts. At most, existing non-powered techniques, such as one-way valves, prevent backflow of blood from higher pressure regions to lower pressure regions.

It may be desirable to provide a technique for delivering blood from a lower pressure region to a higher pressure region without requiring a power source and/or moving parts. Such a technique would not be prone to failure due to failure of the power source, and may be less susceptible to wear and failure compared to techniques requiring moving parts. In some applications, this may reduce secondary procedures and/or reduce morbidities associated with failures of power sources and/or moving parts.

The Venturi effect is the reduction in pressure of a fluid flowing in a conduit as the fluid flows through a narrow segment of the conduit. The pressure of the fluid in the conduit upstream from the narrow segment is greater than the pressure of the fluid in the narrow segment. This is due, at least in part, to an increase in velocity of the fluid as it passes through the narrow segment. The Venturi effect may be utilized to deliver blood from a lower pressure region to a higher pressure region without the use of a pump or other powered device in some applications.

According to embodiments of the present disclosure, a conduit with a narrow segment may be coupled to two portions of a higher pressure region, thus creating a parallel flow path for the blood in the higher pressure region. Blood may be delivered from a lower pressure region to the narrow segment of the conduit. The narrow segment may provide a pressure drop (e.g., due to the Venturi effect) that allows the blood to flow from the lower pressure region to the higher pressure region. In some applications, devices and methods disclosed herein may utilize the Venturi effect, at least in part, to deliver oxygenated blood from the left atrium or pulmonary vein to the aorta without a power source and/or moving parts. In some applications, this may increase the effective oxygenated blood flow through the aorta. In some applications, this may at least partially compensate for low ejection fraction of the left ventricle.

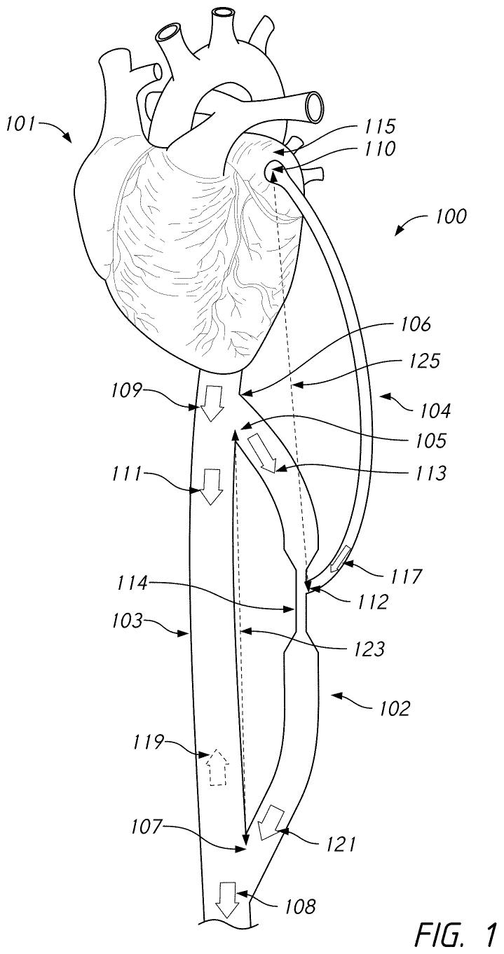

FIG. 1 is an illustration of a device coupled to a heart and an aorta according to at least one embodiment of the present disclosure. Device 100 may be coupled to a higher pressure region and a lower pressure region. In the example shown in FIG. 1, device 100 is coupled to a left atrium 115 of a heart 101 and an aorta 103. In this example, the left atrium 115 may be a lower pressure region and the aorta 103 may be a higher pressure region. However, the lower and higher pressure regions may be different in other examples. For example, the lower pressure region may be a pulmonary vein in other examples.

The device 100 may include a first conduit 102 and a second conduit 104. The first conduit 102 may have a first end 106 fluidly coupled to the aorta 103 at a first site 105 and a second end 108 fluidly coupled to the aorta 103 at a second site 107. The first site 105 may be upstream with respect to blood flow through the aorta (indicated by arrow 109) and/or proximate the heart 101 relative to second site 107, which may be downstream and/or distal to the heart 101 relative to first site 105. In some examples, a distance 123 between the first end 106 and the second end 108 along the aorta 103 may be 5-20 centimeters. At least some blood flowing through the aorta 103 may be diverted through the first conduit 102 as indicated by arrow 113. In some embodiments, about 15-50% of the blood flow of the aorta 103 may be diverted through the first conduit 102. In some embodiments, blood flow of about 9-30 cubic centimeters per second may be diverted through the first conduit 102. The diverted blood may flow through the first conduit 102 and rejoin the un-diverted blood (indicated by arrow 111) in the aorta 103 proximate the second end 108 (indicated by arrow 121). Thus, the first conduit 102 may provide an alternate, or parallel, flow path for blood flow for at least a portion of the aorta 103.

The second conduit 104 may have a first end 110 fluidly coupled to the left atrium 115 and a second end 112 coupled to the first conduit 102 (the first end 110 and second end 112 of the second conduit 104 may also be referred to as third and fourth ends, respectively). The second conduit 104 may divert at least a portion of the blood in the left atrium 115 to the first conduit 102. In some embodiments, about 5-33% of the blood flow of the left atrium 115 may be diverted through the second conduit 104. In some embodiments, the blood flow diverted through the second conduit 104 may be about 3-20 cubic centimeters per second. The second end 112 of the second conduit 104 may be coupled to a narrow segment 114 of the first conduit 102 disposed between the first end 106 and the second end 108 of the first conduit 102. In some examples, a distance 125 between the first end 110 and the second end 112 of the second conduit 104 may be between 10 and 25 centimeters. The distance 125 may be the same as a length of the second conduit 104 or may be different. The narrow segment 114 may have a diameter that is narrower than a diameter of the first conduit 102 at the first end 106 and the second end 108. Based, at least in part, on the Venturi effect, the fluid pressure within the narrow segment 114 may be less than the fluid pressure in the aorta 103 and other portions of the first conduit 102. The pressure in the narrow segment 114 may be low enough such that blood is drawn from the second conduit 104 into the first conduit 102 as indicated by arrow 117. In some embodiments, the pressure in the narrow segment 114 may be lower than the pressure in the second conduit 104.

The blood from the left atrium 115 diverted through the second conduit 102 may be combined with the blood of the aorta 103 diverted through the first conduit 102. The blood diverted from the left atrium 115 and the blood diverted from the aorta 103 may be combined with the un-diverted blood in the aorta 103 at second site 107 proximate the second end 108 of the first conduit 102. Thus, the blood diverted from the left atrium 115 may bypass the left ventricle of the heart 101. In some applications, bypassing the left ventricle with device 100 may at least partially compensate for poor ejection fraction of the left ventricle.

During ventricular diastole, blood in the aorta 103 may temporarily reverse flow as indicated by arrow 119. If the reverse blood flow in the aorta 103 were to push blood from the first conduit 102 into the second conduit 104 and into the left atrium 115, this could cause damage to the heart 101. Accordingly, back flow of blood into the left atrium 115 is undesirable. In some embodiments, the Venturi effect may obviate the need for valves or other components for preventing backflow of blood from the aorta 103 and/or first conduit 102 into the second conduit 104 and/or left atrium 115. During ventricular diastole, pressure in the aorta 103 may decrease and the pressure in the narrow segment 114 may be lower during ventricular diastole than during ventricular systole. Due, at least in part, to the Venturi effect in the narrow segment 114, the pressure in the second conduit 104 may be equal to or greater than the pressure in the narrow segment 114 during ventricular diastole. The higher pressure in the second conduit 104 compared to the narrow segment 114 may prevent backflow of blood into the left atrium 115 in some embodiments.

Although FIG. 1 shows the second conduit 104 coupled to narrow segment 114 at a side of the narrow segment 114 distal to the aorta 103, the second conduit 104 may be coupled at any location around the circumference of narrow segment 114. Furthermore, while FIG. 1 shows the first conduit 102 coupled to the aorta 103 on a side proximate the left ventricle 115, the first conduit 102 may be coupled to the aorta 103 at any location around the circumference of the aorta 103.

In some embodiments, the first conduit 102 and second conduit 104 may be of unitary construction. That is, device 100 may be formed as a single piece or unit. In some embodiments, the device 100 may be free of seams or joints between the first conduit 102 and the second conduit 104. In some embodiments, the device 100 may be constructed of a biocompatible material. The biocompatible material may be polytetrafluoroethylene (e.g., Teflon). In some embodiments, the device 100 may include one or more coatings on the interior and/or exterior surfaces of the conduits 102, 104, such as anti-coagulant and/or anti-fouling coatings.

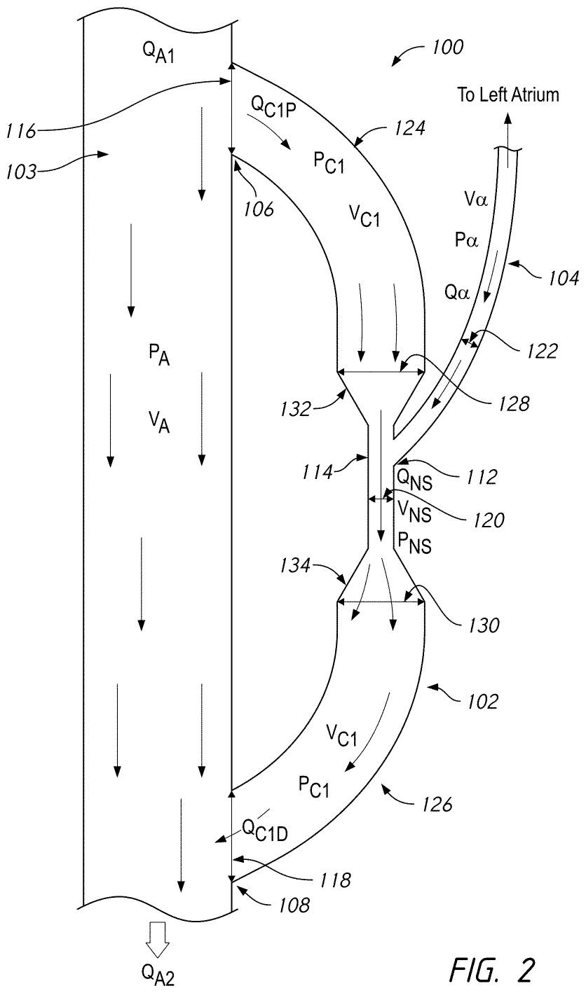

FIG. 2 is an illustration of a portion of the device illustrated in FIG. 1. FIG. 2 is a magnified view of the device 100 near the narrow segment 114. While a portion of the aorta 103 is shown for context, the heart 101 and the first end 110 of the second conduit 104 are not shown in FIG. 2. In some embodiments, the first conduit 102 may have a first diameter 116 at the first end 106 and a second diameter 118 at the second end 108. In some embodiments, the diameters 116 and 118 may be the same (which, as used herein, may mean, e.g., equal, substantially equal). In some embodiments, a segment 124 of the first conduit 102 between the first end 106 and the narrow segment 114 may have the same diameter as diameter 116 until a transition segment 132. Thus, diameter 128 may be the same as diameter 116. Similarly, a segment 126 of the first conduit 102 between the second end 108 and the narrow segment 114 may have the same diameter as diameter 118 until a transition segment 134. Diameter 130 may be the same as diameter 118. As used herein, diameter refers to an inner diameter of the conduits 102, 104. In some embodiments, diameter 116, 118, 128, and/or 130 may be to the same as a diameter of the aorta 103. In some embodiments, a length of segment 124 and a length of segment 126 may be the same. In some embodiments, the narrow segment 114 may be disposed equidistant between the first end 106 and the second end 108. However, in other embodiments, segments 124 and 126 may have different lengths. In some embodiments, segment 114 may be disposed closer to the first end 106 than the second end 108, or closer to the second end 108 than the first end 106.

In some embodiments, the narrow segment 114 may have a diameter 120 that is less than at least one of diameter 116 and diameter 118. In some embodiments, the diameter 120 may be 15% to 35% (inclusive) of diameter 116 and/or diameter 118. In some embodiments, the narrow segment 114 may have the same diameter for the length of the narrow segment 114. The second conduit 104 may have a diameter 122. In some embodiments, the second conduit 104 may have the same diameter from the first end 110 (shown in FIG. 1) to the second end 112. In some embodiments, the diameter 122 may be to the same as the diameter 120 of the narrow segment 114.

The difference between diameter 120 of the narrow segment 114 and the other portions of the first conduit 102 may generate a Venturi effect when fluid, such as blood, flows through the first conduit 102. The Venturi effect may be described by:

P_{A}−P_{NS}=(ρ_{B}/2)(V_{NS}²−V_{A}²) Equation 1

Where P_{A} is the pressure in the aorta, P_{NS} is the pressure in the narrow segment 114, ρ_{B} is the density of blood, V_{NS} is the velocity of blood in the narrow segment 114, and V_{A} is the velocity of blood in the aorta 103. As provided by Equation 1, as velocity in the narrow segment 114 increases relative to the velocity in the aorta 103, the pressure in the narrow segment 114 decreases relative to the pressure in the aorta 103. Velocity of a fluid through a cylindrical (or substantially cylindrical) conduit, such as conduit 102, conduit 104, and/or aorta 103 is provided by:

$$V = \frac{Q}{A} \qquad Equation 2$$

Where Q is flow and A is the area of a cross section of the conduit. The area may be provided by:

A=π(d/2)² Equation 3

Where d is the diameter of the conduit. As shown by Equations 2-3, the velocity is inversely proportional to the diameter of the conduit. Thus, as the diameter of the conduit decreases, the velocity increases, which in turn leads to a decrease in pressure. Based on the underlying physics of the Venturi effect and generally accepted principles of physiology, the following assumptions may be made:

V_{C1}=V_{A} Equation 4

P_{C1}=P_{A} Equation 5

V_{NS}>V_{A} Equation 6

P_{NS}<P_{A} Equation 7

Q_{C1P}<Q_{A1} Equation 8

Q_{C1D}=Q_{C1P}+Q_{α}=Q_{NS} Equation 9

Q_{A2}=Q_{A1}+Q_{α} Equation 10

P_{α}=P_{LA}+ρ_{B}gh Equation 11

V_{NS}A_{NS}=V_{A}A_{A} Equation 12

Where V_{C1} is the velocity of blood through the first conduit 102 at the first end 106 and the second end 108, P_{C1} is the pressure in the first conduit 102 at the first end 106 and the second end 108, Q_{C1P} is the blood flow (e.g., cubic centimeters per second) through the first conduit 102 in the segment 124 proximate the heart 101 (shown in FIG. 1), Q_{A1} is the blood flow through the aorta 103 upstream from the first end 106 of the first conduit 102, Q_{C1D} is the blood flow through the first conduit 102 in segment 126 distal to the heart 101, Q_{α} is the blood flow through the second conduit 104, Q_{NS} is the blood flow through the narrow segment 114, and Q_{A2} is the blood flow through the aorta 103 downstream from the second end 108 of the first conduit 102. Further, P_{α} is the pressure in the second conduit 104, P_{LA} is the pressure in the left atrium 115, g is the acceleration due to gravity, h is the distance between the site where the first end 110 of the second conduit 104 is coupled to the left ventricle 115 and the site where the second end 112 of the second conduit 104 is coupled to the narrow segment 114, A_{NS} is an area of a cross section of the narrow segment 114, and A_{A} is an area of a cross section of the aorta 103.

In some applications, the goal may be to maximize Q_{α} and the diameters of the various segments 114, 124, 126 of first conduit 102 and/or the diameter 122 of the second conduit 104 may be adjusted relative to one another accordingly based, at least in part, on Equations 1-12 above. Returning to Equation 12, V_{NS} may be provided by:

$${V}_{NS} = {V}_{A} \frac{{A}_{A}}{{A}_{NS}} = {{V}_{A} ( \frac{{r}_{A}}{{r}_{NS}} )}^{2} \qquad Equation 13$$

Where r_{A} is the radius (e.g., half of the diameter) of the aorta 103 and r_{NS} is the radius (e.g., half of the diameter 120) of the narrow segment 114. In some embodiments, the diameter of the aorta 103 may be the same as the diameter 116, 128, 130, and/or 118 of the first conduit 102. Plugging Equation 13 into Equation 11, provides:

$$\frac{2 ( {P}_{DA} - {P}_{NS} )}{{ρ}_{B}} = {V}_{A}^{2} [ {( \frac{{r}_{A}}{{r}_{NS}} )}^{4} - 1 ] \qquad Equation 14$$

To allow blood to flow from the lower pressure region (e.g., left atrium) to the higher pressure region (e.g., aorta), the pressure in the narrow segment 114 must be less than the pressure P_{α} of the second conduit 104:

P_{NS}<P_{LA}+ρ_{B}gh Equation 15

By plugging Equation 15 into Equation 14, it can be derived:

$$1 + \frac{2 ( {P}_{A} - {P}_{LA} - {ρ}_{B} gh )}{{ρ}_{B} {V}_{A}^{2}} = {( \frac{{r}_{A}}{{r}_{NS}} )}^{4} \qquad Equation 16$$

To simplify:

$$m = \frac{2 ( {P}_{A} - {P}_{LA} - {ρ}_{B} gh )}{{ρ}_{B} {V}_{A}^{2}} \qquad Equation 17$$

Where m represents the portion of the pressures of the device 100 and surrounding anatomy that does not depend on diameters of the conduits or anatomy. In some applications, m may represent pressures that cannot be controlled by adjusting various dimensions of the device 100 and/or surrounding anatomy. The simplified version of Equation 16 is given by:

$$1 + m = {( \frac{{r}_{A}}{{r}_{NS}} )}^{4} \qquad Equation 18$$

Thus, the ratio of the radius (e.g., half the diameter 120) of the narrow segment 114 and the radius of the aorta 103 that provides for a sufficient Venturi effect is provided by:

$$\frac{{r}_{NS}}{{r}_{A}} < {( \frac{1}{1 + m} )}^{1 / 4} \qquad Equation 19$$

Thus, Equation 19 may be used to determine a suitable radius, and thus a suitable diameter 120, for the narrow segment 114 when the diameter 116, 128, 130, and/or 118 of the first conduit 102 is to the same as the diameter of the aorta 103.

In embodiments where the device 100 is coupled to a heart 101 and an aorta 103, as in the examples shown in FIGS. 1-2 and as will be described in a congestive heart failure patient example below, in some applications, the various diameters of the device 100 may be based, at least in part, on a condition of the subject, such as the ejection fraction of the subject's left ventricle. For example, the diameter 122 of the second conduit 104 may be increased to divert more blood from the left atrium 115 (shown in FIG. 1) in cases where the ejection fraction is very low. In another example, the diameters 116, 118, 128, and 130 may be increased when more blood flow through the first conduit 102 is needed to provide sufficient draw (e.g., via the Venturi effect) of the blood from the second conduit 104 into the aorta 103.

An illustrative example for selecting suitable diameters for the various portions of device 100 for a patient with congestive heart failure is provided herein. However, the disclosure is not limited to this particular example. A patient with congestive heart failure may have the following values:

P_{A}=80 mmHg=10,666 Pa Equation 20

P_{LA}=11 mmHg=2,000 Pa Equation 21

V_{A}=0.55 m/s Equation 22

h=0.15 m Equation 23

ρ_{B}=1060 kg/m³ Equation 24

g=9.81 ms² Equation 25

Plugging the above example values into Equation 17, m is calculated to be approximately 45.32. Using this value for m in Equation 19:

$$\frac{{r}_{NS}}{{r}_{A}} < 0.363 \qquad Equation 26$$

The average radius of an aorta for a patient suffering from congestive heart failure is in the range of about 10 mm to 18 mm. Thus, when r_{A}=10 mm, r_{NS}<3.63 mm and when r_{A}=17.5 mm, r_{NS}<6.36 mm based on Equation 26 in this example. Thus, a device with a narrow segment having a diameter of about 7 mm may be selected for a congestive heart failure patient who has an aorta with a diameter of about 20 mm and a device with a narrow segment having a diameter of about 12 mm may be selected for a congestive heart failure patient who has an aorta with a diameter of about 35 mm.

In some applications, a biofilm may develop over time on an interior surface of the narrow segment 114 and/or other portions of the device 100. The biofilm may include lipids, proteins, and/or microorganisms. While one or more biofilm-resistant coatings may be applied to device 100 and/or device 100 may be constructed of a material that is resistant to biofilms, in some cases, a biofilm may still develop over time. The biofilm may effectively reduce the diameter 120 of the narrow segment 114. Thus, in some embodiments, the diameter 120 of the narrow segment 114 near the high end of the acceptable range may be selected. Returning to the congestive heart failure example discussed above, r_{NS} may have a value close to 3.63 mm when the aorta 103 has a radius of about 10 mm and r_{NS} may have a value close to 6.36 when the aorta 103 has a radius of about 17.5 mm.

In some applications, images of the patient's aorta may be acquired (e.g., angiogram, magnetic resonance imaging) in order to determine the diameter of the aorta. The various diameters of device 100, such as the diameters 116, 118, 128, 130 of the first conduit 102, diameter 122 of the second conduit 104, and diameter 120 of the narrow segment 114 may be selected based on the measurements obtained from the patient. In some embodiments, the device 100 may be custom-made based on the anatomy of the patient in addition to or instead of the condition of the patient.

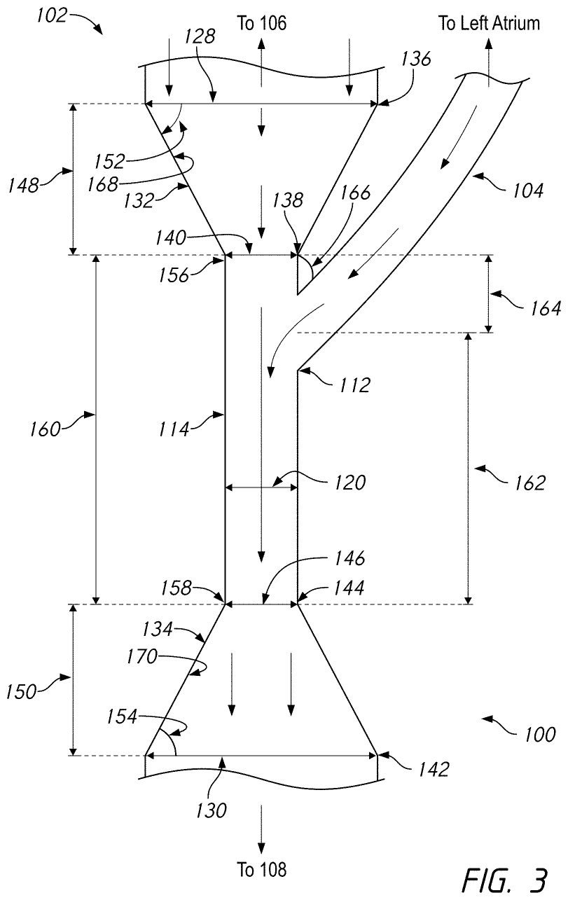

FIG. 3 is an illustration of a portion of the device illustrated in FIGS. 1 and 2. FIG. 3 is a magnified view of the device 100 near the narrow segment 114. The first end 106 and second end 108 of the first conduit 102 and the first end 110 of the second conduit 104 are not shown in FIG. 3. In some embodiments, the first conduit 102 may gradually change diameters from diameters 128 and 130 to the diameter 120 of the narrow segment 114 in transition segments 132 and 134. The transition segments 132 and 134 may have truncated conical structures with diameters that vary linearly over distance in some embodiments, such as the one shown in FIG. 3. However, in other embodiments, the transition segments 132 and 134, independently of each other, may have different structures with diameters that change differently with distance (e.g., hyperbolic, polynomial).

In the example shown in FIG. 3, transition segment 132 may have a first end 136 proximate the first end 106 of the first conduit 102 (shown in FIGS. 1-2) that has a diameter 128 and a second end 138 proximate the narrow segment 114 that has a diameter 140 (the first end 136 and the second end 138 of the transition segment 132 may also be referred to as fifth and sixth ends, respectively). In some embodiments, diameter 140 may be the same as diameter 120 of the narrow segment 114. The transition segment 132 may have a length 148. The length 148 and difference between diameters 128 and 140 may provide for an angle 152 between an interior wall 168 of the transition segment 132 and a plane perpendicular to the flow of fluid through the conduit 102. As the difference between diameters 128 and 140 increases, the angle 152 decreases when the length 148 is held constant. As the length 148 is increased, the angle 152 increases when the difference between diameters 128 and 140 are held constant. In some applications, a longer length 148 and greater angle 152 may be desirable for reducing cardiac stress.

Transition segment 134 may have a first end 142 proximate the second end 108 of the first conduit 102 (shown in FIGS. 1-2) that has a diameter 130 and a second end 144 proximate the narrow segment 114 that has a diameter 146 (the first 142 and second 144 ends of the transition segment 134 may also be referred to as seventh and eighth ends, respectively). In some embodiments, diameter 146 may be the same as diameter 120 of the narrow segment 114. The transition segment 134 may have a length 150. The length 150 and difference between diameters 130 and 146 may provide for an angle 154 between an interior wall 170 of the transition segment 134 and a plane perpendicular to the flow of fluid through the conduit 102. As the difference between diameters 130 and 146 increases, the angle 154 decreases when the length 150 is held constant. As the length 150 is increased, the angle 154 increases when the difference between diameters 130 and 146 are held constant. In some applications, a longer length 150 and greater angle 154 may be desirable for reducing cardiac stress.

In some embodiments, the dimensions of the transition segments 132 and 134 may be the same. For example, diameters 128 and 130 may be the same, diameters 140 and 146 may be the same, and lengths 148 and 150 may be the same. However, in some embodiments, the transition segments 132 and 134 may have one or more dimensions that differ. For example, diameters 128 and 130 may be different. In another example, lengths 148 and 150 may be different. In some applications, length 150 may be greater than 148, which may assist in preventing back flow of blood from the aorta 103 into the second conduit 104 during ventricular diastole.

In some embodiments, the narrow segment 114 may have a first end 156 proximate the first transition segment 132 (which is proximate the first end 106 of the first conduit 102) and a second end 158 proximate the second transition segment 134 (which is proximate the second end 108 of the first conduit 102) and have a length 160 (the first 156 and second 158 ends of the narrow segment 114 may also be referred to as ninth and tenth ends, respectively). In some embodiments, the second end 112 of the second conduit 104 may be coupled to the narrow segment 114 at a location equidistant from the first end 156 and the second end 158. However, in other embodiments, the second end 112 of the second conduit 104 may be coupled to the narrow segment 114 at a location closer to the first end 156 or closer to the second end 158 of the narrow segment 114. In the example shown in FIG. 3, the second end 112 of the second conduit 104 is coupled to the narrow segment 114 at a location closer to the first end 156. Thus, a length 164 between the first end 156 and a center of the second end 112 of the second conduit 104 is less than a length 162 between the second end 158 and the center of the second end 112 of the second conduit 104. In some applications, coupling the second end 112 of the second conduit 104 closer to the first end 156 may assist in preventing back flow of blood from the aorta 103 into the second conduit 104 during ventricular diastole.

In some embodiments, the second end 112 of the second conduit 104 may be coupled to the narrow segment 114 at an angle 166. In some embodiments, such as the one shown in FIG. 3, the angle 166 is less than 90 degrees. In some applications, 45 degrees or less may be desirable for angle 166. In some applications, a smaller angle 166 may reduce turbulent flow proximate where the second end 112 is coupled to the narrow segment 114. In some applications, the angle 166 may be based, at least in part, on an orientation between the left atrium 115 (shown in FIG. 1) and where the first conduit 102 is coupled to the aorta 103. For example, the angle 166 may be smaller when the device 100 is located at a position along the aorta 103 relatively more distal to the heart 101 and left atrium 115 and larger when the device 100 is located at a position along the aorta 103 relatively more proximate to the left atrium 115.

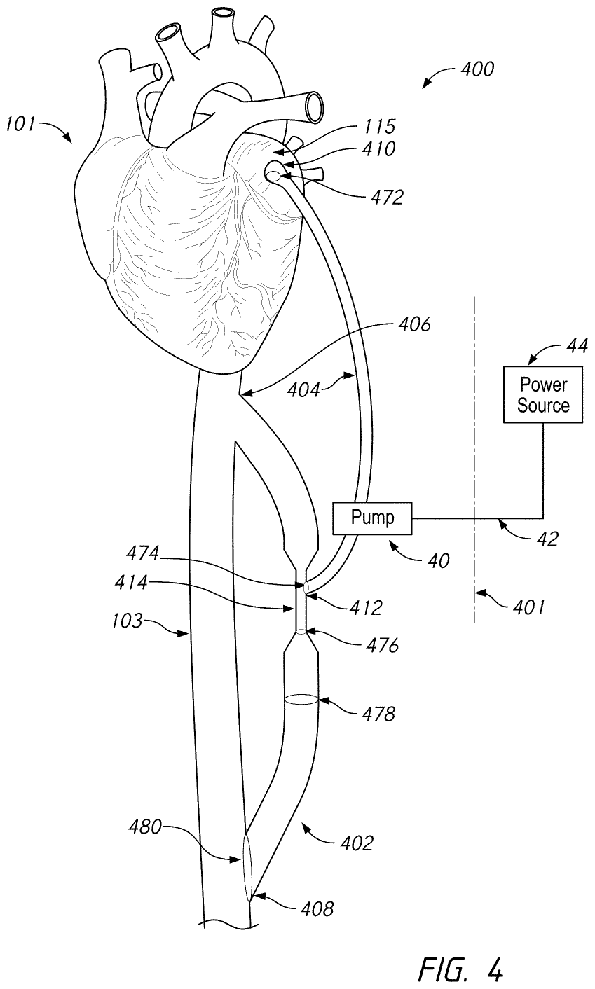

FIG. 4 is an illustration of a device coupled to a heart and an aorta according to at least one embodiment of the present disclosure. The device 400 may be substantially the same as device 100 shown in FIG. 1-3 except that while device 100 is free of any valves, device 400 may include one or more one-way valves and/or a pump 40. Accordingly, for brevity, the features of device 400 that are the same as device 100 will not be discussed herein. Same reference numerals between FIGS. 1-3 and 4 refer to the same features and reference numerals having the same lesser digits refer to like features between device 100 and device 400.

Although the Venturi effect may inhibit back flow of blood from a higher pressure region, such as aorta 103, into a lower pressure region, such as left atrium 115, through the second conduit 404, in some applications, the Venturi effect may not be sufficient to prevent back flow. In these applications, one or more one-way valves and/or a pump may be included in the device 400 to prevent or reduce back flow of blood. While FIG. 4 illustrates five valves 470-478, this is merely to show example suitable locations for valves, and device 400 may have fewer valves or only one valve in some embodiments. In some applications, the Venturi effect may not be sufficient to draw a desired volume of blood from the left atrium 115 into the aorta 103. In these applications, a pump may be provided to increase the volume of blood that bypasses the left ventricle.

A valve 472 may be located at the first end 410 of the second conduit 404 in some embodiments. The valve 472 may be located proximate an opening in the left ventricle 115 that fluidly couples the left ventricle 115 to the second conduit 404. In some embodiments, a valve 474 may be located at the second end 412 of the second conduit 404 where the second conduit 404 is fluidly coupled to the narrow segment 414. In other embodiments, a valve may be located anywhere along the length of the second conduit 404.

In some embodiments, valve 476 may be located at an end of the narrow segment 414 proximate the second end 408 of the first conduit 402. In other embodiments, a valve may be located anywhere along the length of narrow segment 414 that is downstream (during ventricular systole) of the second end 412 of the second conduit 404. In some embodiments, a valve 478 may be located downstream of the narrow segment 414. In some embodiments, a valve 480 may be located proximate the second end 408 at an opening in the aorta 103 that fluidly couples the aorta 103 to the first conduit 402. In other embodiments, a valve may be located anywhere along the length of the first conduit between the narrow segment 414 and the second end 408. The valves 472-480 may include any suitable one-way valve. For example, mono-cuspid valves or tri-leaflet valves may be used. The valves 472-480 may be the same or different.

Optionally, device 400 may include a pump 40. The pump 40 may be disposed at a location between the first end 410 and the second end 412 of the second conduit 404 prior to where the second conduit 404 joins the narrow segment 414. While the pump 40 is shown located closer to the second end 412 in FIG. 4, in other embodiments, the pump 40 may be located closer to the first end 410. In some embodiments, the pump 40 may be included in addition to one or more of the valves 472-480. In other embodiments, the pump 40 may be included in device 400 and one or more of the valves 472-480 may be omitted.

In some embodiments, the pump 40 may be a continuous flow pump. In some embodiments, the pump 40 may be an axial flow pump. In some embodiments, the pump 40 may be a centrifugal pump. Examples of suitable pumps that may be used include the HeartMate™ II or III Left Ventricular Assist Device (LVAD) (Abbott, Abbott Park, Ill.) and Impella Heart Pump (Abiomed, Danvers, Mass.). However, any other suitable pump may be used. In some embodiments, the pump 40 may be powered by an external power source 44. In some embodiments, the pump 40 is coupled to the power source 44 by a conductive line 42 that runs through the skin 401, similar to existing left ventricular assist devices. In some embodiments, the external power source may be a battery.

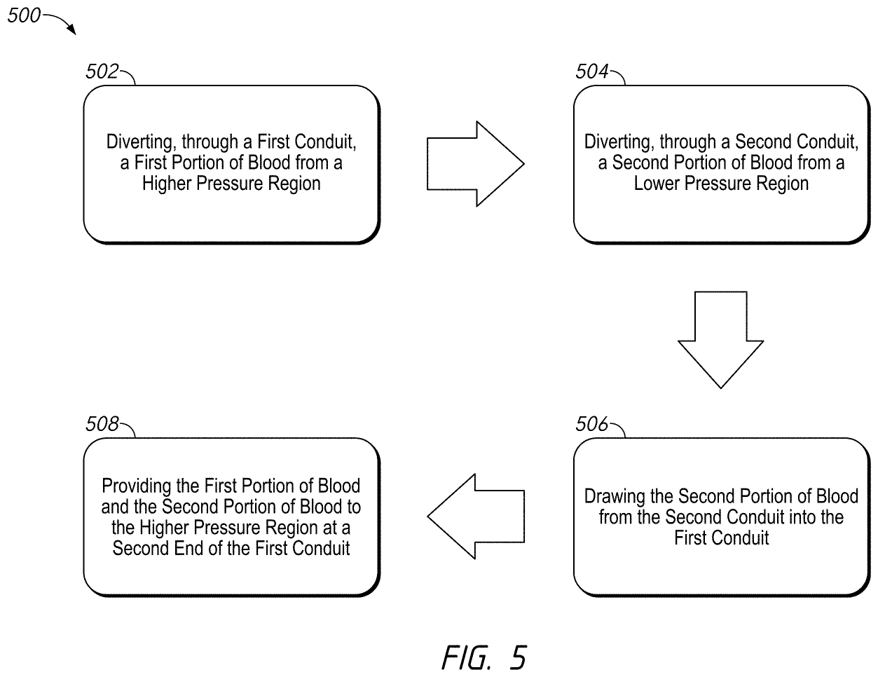

FIG. 5 is a flow chart of a method according to at least one embodiment of the present disclosure. Method 500 may be a method for delivering blood from a lower pressure region to a higher pressure region. In some embodiments, the method 500 may be performed by a device, such as device 100 in FIGS. 1-3 and/or device 400 in FIG. 4.

As indicated at block 502, a first portion of blood may be diverted from the high pressure region into a first conduit (e.g., 102, 402). In some embodiments, a first end (e.g., 106, 406) of the first conduit is fluidly coupled to the higher pressure region (e.g., aorta 103) at a first location (e.g., site 105) and a second end (e.g., 108, 408) of the first conduit is fluidly coupled to the higher pressure region at a second location (e.g., site 107) downstream of the first location. The first portion of blood may flow through at least a portion of the first conduit from the first end toward the second end.

A second portion of blood may be diverted from the lower pressure region (e.g., left atrium 115) into a second conduit (e.g., 104, 404) as indicated by block 504. Although block 504 is shown after block 502, in some embodiments, block 504 may be performed before block 502 or block 504 and block 502 may be performed concurrently, at least in part. In some embodiments, an end (e.g., 110, 410) of the second conduit is fluidly coupled to the lower pressure region and another end (e.g., 112, 412) of the second conduit is fluidly coupled to a narrow segment (e.g., 114, 414) of the first conduit. The narrow segment is disposed between the first end and the second end. The second portion of blood may flow through the second conduit from the end coupled to the lower pressure region toward the end coupled to the narrow segment. The blood that is not diverted from the lower pressure region may flow to the higher pressure region. For example, blood not diverted from the left atrium into second conduit may be provided to a left ventricle of the heart and pumped by the left ventricle into the aorta.

Optionally, in some embodiments at block 504, blood may be diverted from the lower pressure region into the second conduit due at least in part, by drawing blood from the lower pressure region into the second conduit with a pump (e.g., pump 40).

As indicated by block 506, the second portion of blood may be drawn from the second conduit into the first conduit. In some embodiments, the narrow segment may generate a Venturi effect to help draw the second portion of blood. The second portion of blood may join the first portion of blood at the narrow segment.

Optionally, in some embodiments at block 506, blood may be drawn from the second conduit into the first conduit due at least in part, by pumping blood from the second conduit into the first conduit with a pump (e.g., pump 40).

The first portion of blood and the second portion of blood may be provided to the higher pressure region at the second end of the first conduit as indicated by block 508. The combined first portion and second portion of blood may be combined with a portion of blood in the high pressure region that was not diverted into the first conduit (e.g., a portion of blood flowing through the aorta) at the second end of the first conduit.

In some embodiments, the lower pressure region may include a left atrium of a heart and the higher pressure region may include an aorta. In these embodiments, the method 500 may further include coupling the first end and the second end of the first conduit to the aorta and coupling the third end of the second conduit to the left atrium. The conduits may be coupled by sutures, clamps, and/or any other suitable coupling techniques.

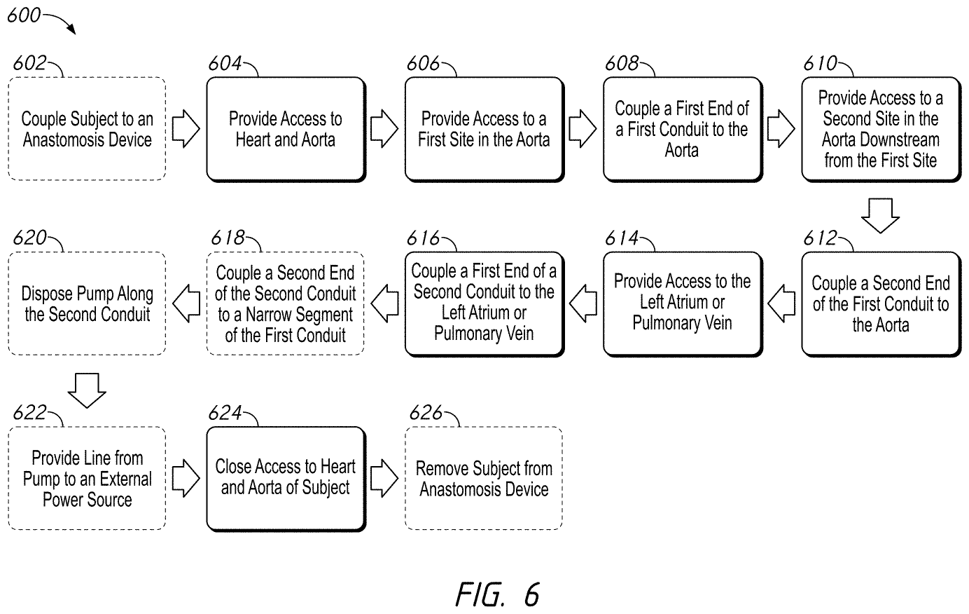

FIG. 6 is a flow chart of a method according to at least one embodiment of the present disclosure. Method 600 may be a method for installing (e.g., implanting) a device to deliver blood from a lower pressure region to a higher pressure region, such as device 100 in FIGS. 1-3 and/or device 400 in FIG. 4. The device may be implanted in a subject, such as a person suffering from congestive heart failure. In some applications, method 600 may be performed, at least in part, by a cardiac surgeon and/or other clinician. Thus, detailed explanations of procedures and surgical techniques within the skill of such clinicians (e.g., anesthesia, operation of a cardiac bypass machine) are not be provided.

Optionally, as indicated at block 602, a subject may be coupled to an anastomosis device. Any suitable device and/or technique, such as a conduit anastomosis, may be used. In some implementations, the subject is coupled to a cardiac bypass machine. However, in some embodiments, the subject may not be coupled to a cardiac bypass machine and other techniques and/or devices may be used. Access to a heart (e.g., 101) and aorta (e.g., 103) of the subject may be provided as indicated by block 604. For example, an incision may be made. Any appropriate technique for making the incision may be used. In some embodiments, multiple incisions may be made. The location and/or number of incisions may be based, at least in part, on the chosen surgical method (e.g., laparoscopic, open chest).

As indicated at block 606, access to a first site (e.g., 105) in the aorta may be provided and a first end (e.g., 106, 406) of a first conduit (e.g., 102, 402) may be coupled to the aorta at the first site to fluidly couple the first conduit to the aorta as indicated by block 608. Access to a second site (e.g., 107) in the aorta may be provided downstream from the first site and a second end (e.g., 108, 408) of the first conduit may be coupled to the aorta at the second site as indicated by blocks 610 and 612. In some embodiments, the first and second access sites in the aorta may be based, at least in part, on a condition of the patient (e.g., partial ejection fraction), location and/or size of the aorta, and/or the relative locations of the left atrium and aorta to one another. In some embodiments, the first and second sites may be selected based, at least in part, on a size of the device, a diameter of the first conduit, and/or a length of the first conduit.

As shown in blocks 614 and 616, access may be provided to the left atrium (e.g., 115) or a pulmonary vein and a first end (e.g., 110, 410) of a second conduit (e.g., 104, 404) may be coupled at the access site to fluidly couple the second conduit to the left atrium or pulmonary vein. In some embodiments, the location of the access site in the left atrium or pulmonary vein may be based, at least in part, on the amount of blood to be diverted, a diameter of the second conduit, a length of the second conduit, the location where the first conduit is installed, and/or anatomical limitations (e.g., a suitable distance away from the pulmonary vein and/or heart valves, a location where the pulmonary vein is of suitable thickness). In some examples, an incision may be made to provide access to the aorta and left atrium. A scalpel, cauterizing scalpel, or other suitable tool may be used to cut openings into the tissue of the aorta, pulmonary vein, and/or left atrium to fluidly couple these regions to the device. The first conduit may be coupled to the aorta and the second conduit may be coupled to the left atrium or pulmonary vein by any suitable technique. For example, sutures, staples, and/or clamps may be used. Optionally, in embodiments when the first and second conduits are not formed as an integral unit, as indicated by block 618, a second end (e.g., 112, 412) of the second conduit may be coupled to a site along a narrow segment (e.g., 114, 414) of the first conduit.

Optionally, in embodiments that include a pump (e.g., pump 40 of device 400 shown in FIG. 4), the pump may be disposed along the second conduit as indicated by block 620. However, in some embodiments, the pump may already be disposed along the second conduit. In some embodiments, a line (e.g., line 42) between the pump and an external power supply (e.g., power source 44) may be provided as indicated by block 622. In some examples, the line may pass through the skin (e.g., via a port) to couple to the external power supply.

The order of the blocks 606-622 are provided merely as an example, and the blocks 606-622 may be arranged in different orders. For example, blocks 610 and 612 may be performed prior to blocks 606 and 608. In some embodiments, blocks 614 and 616 may be performed before blocks 610 and 612 and/or before blocks 606 and 608. In some embodiments, block 618 may be performed prior to blocks 606, 608, 610, 612, 614, and/or 616. In some embodiments, all three incisions indicated by blocks 606, 610, and 614 may be performed prior to all of the coupling indicated by blocks 608, 612, and 616.

Although not shown in FIG. 6, in some embodiments, method 600 may optionally include trimming the lengths of the first and/or second conduit prior to coupling. That is, the clinician may finalize the length of the first and/or second conduit during implantation. For example, it may not be possible to obtain precise anatomical measurements of the subject prior to implantation in some applications. Thus, the clinician may not know until the performance of method 600 the precise lengths of the conduits that are required.

After blocks 604-616 (and optionally 602, 618, 620, and/or 622) have been performed, the access to the heart and aorta of the subject may be closed as indicated by block 624. Any suitable technique for closing the access may be used (e.g., sutures, staples, glue). As indicated by block 626, optionally, the subject may be removed from the anastomosis device if block 602 was performed. For example, the subject may be removed from a bypass machine if a bypass machine is used. As noted, in some applications, a bypass machine may not be used. For example, a proximal clampless anastomotic device, or similar device, may be used to perform block 606, 608, 610, 612, 614, and/or 616 and may obviate the need for a bypass machine or other similar device, to perform method 600. An example of a proximal clampless anastomotic device is the PAS-Port System by Cardica, Inc. (Redwood City, Calif.). However, performing method 600 without a bypass machine is not limited to this particular device.

Prior to performing method 600, blood may flow through the lungs to the pulmonary veins back to the heart to fill the left atrium. The left atrium may pump blood into the left ventricle. The left ventricle may then eject the received blood into the aorta. The blood may flow through the aorta to be distributed to smaller vessels throughout the body. After method 600 is performed, some of the blood arriving from the pulmonary veins to the left atrium flows through the second conduit to the narrow segment. The remaining blood in the left atrium may be delivered to the left ventricle. At least some of the blood in the left ventricle may then be pumped into the aorta. Some of the blood may flow through the aorta, similar to before performance of method 600, however, some of the blood may instead flow through the first conduit. The blood may flow through the incision at the first site into the first conduit at the first end and flow through the narrow segment.

As the blood flows through the narrow segment, the velocity of the blood may increase while the fluid pressure of the blood decreases due to the Venturi effect. The decrease in pressure of the blood in the narrow segment may draw the portion of blood that flowed through the second conduit into the narrow segment. Thus, the blood diverted from the aorta may be combined with the blood diverted from the left atrium where the second end of the second conduit is coupled to the narrow segment. The combined blood flows from the first end of the first conduit and the second conduit may flow through the first conduit from the narrow segment to the second end and flow into the aorta through the incision at the second site.

In embodiments, the drawing of blood from the left atrium into the aorta due the Venturi effect may be supplemented by the use of a pump. The pump may increase the volume of blood that may be drawn from the left atrium into the aorta compared to when only the Venturi effect is used.

The blood flowing from the second end of the first conduit may be combined with the blood flowing through the aorta (e.g., the blood not diverted through the first conduit). The blood provided to the aorta at the second end of the first conduit includes the blood pumped by the left ventricle into the aorta and the blood diverted from the left atrium through the device. In some applications, the amount of blood in the aorta available for distribution throughout the body may be greater than the amount of blood available for distribution prior to performance of method 600.

As used herein, the terms “about” and “same” (which, as used herein, may mean, e.g., equal, substantially equal) modifying, for example, the length of a component, a diameter of a component, a volume, a flow rate through at least a portion of a component, and ranges thereof, employed in describing the embodiments of the disclosure, refers to variation in the numerical quantity that can occur, for example, through typical measuring and manufacturing procedures used for making devices; through inadvertent error in these procedures; through differences in the manufacture, implantation, or installation techniques used to provide the devices or carry out the methods, and like proximate considerations. In some instances, the terms “about” and “same” include values up to and including 10% less than and 10% greater than the recited value.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. A device for delivering blood from a lower pressure region to a higher pressure region, the device comprising:
a first conduit comprising:
a first end having a first diameter;
a second end opposite the first end and having a second diameter, wherein the first end and the second end are configured to be fluidly coupled to the higher pressure region; and
a narrow segment disposed between the first end and the second end and having a third diameter, wherein the third diameter is less than the first diameter and the second diameter; and
a second conduit comprising:
a third end configured to be fluidly coupled to the lower pressure region; and
a fourth end opposite the third end, wherein the fourth end is coupled to the first conduit at or near the narrow segment of the first conduit.

2. The device of claim 1, wherein the first conduit further comprises a first transition segment disposed between the first end and the narrow segment, wherein the first transition segment comprises a fifth end proximate the first end and a sixth end proximate the narrow segment, wherein the fifth end has the first diameter and the sixth end has the third diameter.

3. The device of claim 2, wherein the first conduit further comprises a second transition segment disposed between the second end and the narrow segment, wherein the second transition segment comprises a seventh end proximate the second end and an eighth end proximate the narrow segment, wherein the seventh end has the second diameter and the eighth end has the third diameter.

4. The device of claim 3, wherein a length of the second transition segment and a length of the first transition segment are equal.

5. The device of claim 1, wherein the first diameter and the second diameter are equal.

6. The device of claim 1, wherein the second conduit has the third diameter.

7. The device of claim 1, wherein the narrow segment is disposed at a location equidistant between the first end and the second end of the first conduit.

8. The device of claim 1, wherein the narrow segment is disposed at a location closer to the first end than the second end or closer to the second end than the first end of the first conduit.

9. The device of claim 1, wherein the narrow segment comprises a ninth end proximate the first end, and a tenth end proximate the second end, wherein the fourth end of the second conduit is coupled to the first conduit at a location equidistant between the ninth end and the tenth end.

10. The device of claim 1, wherein the narrow segment comprises a ninth end proximate the first end, and a tenth end proximate the second end, wherein the fourth end of the second conduit is coupled to the first conduit at a location closer to the ninth end than the tenth end or closer to the tenth end than the ninth end.

11. The device of claim 1, further comprising a one-way valve disposed proximate the second end of the first conduit, proximate the third end of the second conduit, proximate the fourth end of the second conduit, or proximate an end of the narrow segment proximate the second end of the first conduit.

12. The device of claim 1, wherein the first conduit and the second conduit are free of any valve.

13. The device of claim 1, further comprising a pump disposed along the second conduit between the third end and the fourth end, wherein the pump is configured to draw blood from the lower pressure region and pump blood into the first conduit.

14. A method of delivering blood from a lower pressure region to a higher pressure region, the method comprising:
diverting, through a first conduit, a first portion of blood from the higher pressure region, wherein a first end of the first conduit is fluidly coupled to the higher pressure region at a first location and a second end of the first conduit is fluidly coupled to the higher pressure region at a second location downstream of the first location;
diverting, through a second conduit, a second portion of blood from the lower pressure region, wherein a third end of the second conduit is fluidly coupled to the lower pressure region and a fourth end of the second conduit is fluidly coupled to a narrow segment of the first conduit, the narrow segment disposed between the first end and the second end;
drawing the second portion of blood from the second conduit into the first conduit; and
providing the first portion of blood and the second portion of blood to the higher pressure region at the second end of the first conduit.

15. The method of claim 14, wherein the lower pressure region comprises a left atrium of a heart and the higher pressure region comprises an aorta.

16. The method of claim 15, further comprising:
coupling the first end and the second end of the first conduit to the aorta; and
coupling the third end of the second conduit to the left atrium.

17. The method of claim 14, further comprising inhibiting backflow of the first portion of blood or the second portion of blood with a one-way valve disposed in the first conduit or the second conduit.

18. The method of claim 14, wherein drawing the second portion of blood from the second conduit into the first conduit comprises generating, with the narrow segment, a Venturi effect.

19. The method of claim 18, further comprising inhibiting backflow of the first portion of blood or the second portion of blood with the Venturi effect generated with the narrow segment.

20. The method of claim 14, wherein diverting, through the second conduit, the second portion of blood from the lower pressure region comprises drawing the second portion of blood from the lower pressure region with a pump, and
wherein drawing the second portion of blood from the second conduit into the first conduit comprises pumping the second portion of blood from the second conduit to the first conduit with a pump.
